(19) United States
(12) Patent Application Publication
(10) Pub. No.: US 2014/0277585 A1
(43) Pub. Date: Sep. 18, 2014

(54) SYSTEM AND METHOD FOR POLYMERIC PROSTHETIC LINER PERSPIRATION REMOVAL

(71) Applicant: The Ohio Willow Wood Company, Mount Sterling, OH (US)

(72) Inventors: Christopher T. Kelley, Grandview Heights, OH (US); James M. Colvin, Hilliard, OH (US); Jeffrey A. Denune, Galloway, OH (US); Lonnie L. Nolt, Washington Court House, OH (US)

(21) Appl. No.: 14/214,788

(22) Filed: Mar. 15, 2014

Related U.S. Application Data
(60) Provisional application No. 61/794,948, filed on Mar. 15, 2013.

(51) Int. Cl. A61F 2/78

(52) U.S. Cl. CPC A61F 2/7812
USPC 623/36

(57) ABSTRACT

Various systems and methods for removing perspiration from the interior of a donned polymeric prosthetic liner are disclosed.

### CROSS-REFERENCE TO RELATED APPLICATIONS

This application claims priority from U.S. Provisional Patent Application No. 61/794,948, filed on Mar. 15, 2013, which is incorporated by reference herein in its entirety.

### BACKGROUND

Polymeric prosthetic liners (which may also be referred to herein as “prosthetic liners” or “liners” for brevity) have become the interface of choice among amputees due to various beneficial characteristics thereof. These characteristics include, for example, comfort, security of suspension, protection of the residual limb, and ease of use.

A prosthetic liner has an open (proximal) end via which the liner can be donned, and a closed or essentially closed (distal) end that typically abuts and cushions the end of an amputee's residual limb. Prosthetic liners may be non-suspensory in nature. Non-suspensory liners are commonly referred to as “cushion liners.” Prosthetic liners may optionally be suspensory in nature and may, therefore, include a connecting element that facilitates suspension by mechanical attachment of the liner to a prosthesis. Suspensory liners are commonly referred to as “locking liners.”

In cushion liner form, the area between the socket interior and the liner exterior may be evacuated to draw the liner against the socket. Because the polymeric material of the liner also grips the skin of the residual limb, the socket and associated prosthesis is retained (suspended) on the residual limb. However, if a sufficient amount of perspiration is trapped between the residual limb and the liner interior, then the grip of the liner on the residual limb may be reduced and suspension of the prostheses may be compromised.

In locking liner form, a connecting element of some type, such as a pin receiver, is affixed to the liner at the distal end thereof. The connecting element is adapted to engage and to be retained by a locking mechanism located in a corresponding prosthetic socket. Because the polymeric material grips the skin of the residual limb, the liner may be retained on the residual limb against the torsion (pulling) forces exerted thereon by the weight of the prosthesis that is coupled thereto. Again, however, the presence of a sufficient amount of perspiration between the residual limb and the liner interior can weaken the grip of the liner on the residual limb and compromise suspension of the prosthetic limb.

What is needed is a system with the ability to remove perspiration from the area between a residual limb and a donned prosthetic liner. Additionally, what is needed is a system enhancing the removal of perspiration that does not detrimentally affect the ability of the liner to perform its normal cushioning and suspensory functions.

### SUMMARY

In one embodiment, a system for removing perspiration from an interior of a donned polymeric prosthetic liner is provided, the system comprising: a prosthetic liner having a polymeric body, the polymeric body having an open proximal end for allowing the introduction of a residual limb, a closed or essentially closed distal end opposite the proximal end, an interior, and an exterior; a perspiration wicking fabric disposed along the interior of the polymeric body at the distal end thereof an elastomeric barrier layer disposed along a portion of a proximal side of the perspiration wicking fabric; and a port located at the distal end of the polymeric body and passing through the polymeric body to expose an area of the perspiration wicking fabric.

In another embodiment, a system for removing perspiration from an interior of a donned polymeric prosthetic liner is provided, the system comprising: a prosthetic liner having a polymeric body, the polymeric body having an open proximal end for allowing the introduction of a residual limb, a closed or essentially closed distal end opposite the proximal end, an interior, and an exterior; a perspiration wicking fabric disposed along the interior of the polymeric body at the distal end thereof; an elastomeric barrier layer disposed along a portion of a proximal side of the perspiration wicking fabric; and at least one wicking band having a distal end, the distal end being in communication with the perspiration wicking fabric.

### BRIEF DESCRIPTION OF THE DRAWINGS

The accompanying figures, which are incorporated in and constitute a part of the specification, illustrate various example apparatuses and systems, and are used merely to illustrate various example embodiments.

FIG. 1A illustrates a sectional view of an exemplary embodiment of a polymeric prosthetic liner perspiration removal system.

FIG. 1B illustrates a sectional view of an exemplary embodiment of a polymeric prosthetic liner perspiration removal system.

FIG. 2A illustrates a sectional view of an exemplary embodiment of a polymeric prosthetic liner perspiration removal system.

FIG. 2B illustrates a sectional view of an exemplary embodiment of a polymeric prosthetic liner perspiration removal system.

FIG. 3A illustrates a sectional view of an exemplary embodiment of a polymeric prosthetic liner perspiration removal system.

FIG. 3B illustrates a sectional view of an exemplary embodiment of a polymeric prosthetic liner perspiration removal system.

FIG. 4A illustrates a sectional view of an exemplary embodiment of a polymeric prosthetic liner perspiration removal system.

FIG. 4B illustrates a sectional view of an exemplary embodiment of a polymeric prosthetic liner perspiration removal system.

FIG. 5A illustrates a sectional view of an exemplary embodiment of a polymeric prosthetic liner perspiration removal system.

FIG. 5B illustrates a sectional view of an exemplary embodiment of a polymeric prosthetic liner perspiration removal system.

FIG. 6A illustrates a sectional view of an exemplary embodiment of a polymeric prosthetic liner perspiration removal system.

FIG. 6B illustrates a sectional view of an exemplary embodiment of a polymeric prosthetic liner perspiration removal system.

FIG. 6C illustrates a sectional view of an exemplary embodiment of a polymeric prosthetic liner perspiration removal system.

FIG. 6D illustrates a sectional view of an exemplary embodiment of a polymeric prosthetic liner perspiration removal system.

FIG. 7A illustrates a sectional view of an exemplary embodiment of a polymeric prosthetic liner perspiration removal system.

FIG. 7B illustrates a sectional view of an exemplary embodiment of a polymeric prosthetic liner perspiration removal system.

FIG. 7C illustrates a sectional view of an exemplary embodiment of a polymeric prosthetic liner perspiration removal system.

FIG. 7D illustrates a sectional view of an exemplary embodiment of a polymeric prosthetic liner perspiration removal system.

FIG. 8A illustrates a sectional view of an exemplary embodiment of a polymeric prosthetic liner perspiration removal system.

FIG. 8B illustrates a sectional view of an exemplary embodiment of a polymeric prosthetic liner perspiration removal system.

FIG. 9A illustrates a sectional view of an exemplary embodiment of a polymeric prosthetic liner perspiration removal system.

FIG. 9B illustrates a sectional view of an exemplary embodiment of a polymeric prosthetic liner perspiration removal system.

FIG. 10 illustrates a sectional view of an exemplary embodiment of a polymeric prosthetic liner perspiration removal system.

FIG. 11A illustrates a perspective sectional view of an exemplary embodiment of a polymeric prosthetic liner perspiration removal system.

FIG. 11B illustrates a perspective sectional view of an exemplary embodiment of a polymeric prosthetic liner perspiration removal system.

FIG. 12A illustrates a sectional view of an exemplary embodiment of a polymeric prosthetic liner perspiration removal system.

FIG. 12B illustrates a sectional view of an exemplary embodiment of a polymeric prosthetic liner perspiration removal system.

### DETAILED DESCRIPTION

Liners may be comprised of various polymeric materials, including silicone, urethane, and thermoplastic elastomers (TPE) gels. Liners may be made using various block copolymer and mineral oil gel compositions. Such polymeric materials, particularly block copolymer and mineral oil gel compositions, may provide an optimal level of comfort for most users. T he polymeric materials used to construct such liners may be elastomeric in nature.

Liners may be constructed with an exterior fabric covering that is molded to or otherwise affixed to the underlying polymeric material. This construction may result in a liner having an interior of exposed polymeric material for contacting and cushioning an amputee's residual limb, and an outer layer of fabric for, among other things, increasing the wear resistance of the liner and facilitating donning/doffing and insertion of the liner-covered residual limb into a prosthetic socket.

While polymeric materials may provide amputees with a great deal of cushioning and comfort, the liner-covered residual limb may perspire due to the generally non-permeable nature of the polymeric material. This may be the case whether the polymeric material is a block copolymer, silicone, or another polymeric material.

The amount of perspiration may vary based on a number of factors such as amputee physiology, activity level, and the ambient temperature. In any case, because the polymeric material is not normally permeable to moisture, even a low rate of perspiration production may eventually lead to a substantial collection of perspiration between the residual limb and liner interior. This condition may be exaggerated when the rate of perspiration production is greater, such as during periods of increased physical activity.

The presence of moisture within a donned prosthetic liner may be undesirable for several reasons. First, the moisture may be uncomfortable. Second, from a physiological standpoint, exposing the skin or areas of the skin of a residual limb to moisture for extended periods of time may be unhealthy for the skin. Wet skin may be more susceptible to lesions and/or damage from frictional, shear and pressure forces that may result from socket wear and ambulation. Maintaining a moist environment within a prosthetic liner may also invite the growth of fungus, and the like, which may result in infections and generally create an unsanitary environment.

The presence of moisture within a donned prosthetic liner may be undesirable because it can detrimentally affect prosthesis suspension. In addition to providing a cushioning function, the polymeric material of such a liner may cling to the skin of the residual limb and may seal against the skin. This clinging effect may allow such a liner to function as a suspensory component. Moisture within the liner may reduce the clinging effect.

Embodiments of the system may generally include a polymeric prosthetic liner adapted to permit perspiration to be removed from the interior thereof while in use. To this end, embodiments of the system may also generally include a perspiration wicking fabric located along the liner interior at the distal end thereof. Although the term perspiration wicking “fabric” is used herein for purposes of illustration, it is to be understood that this term is meant to include any material that is capable of collecting and wicking perspiration, whether or not such material technically qualifies as a fabric.

An elastomeric material barrier layer may also be generally present and sized and located to cover a portion of the proximal (interior) side of the perspiration wicking material for the reasons described below. The elastomeric barrier layer material may be the same polymeric material that forms the body of the liner, or it may be a different material. Embodiments of the system may further include a pathway through the liner for removing perspiration therefrom, and various other elements that may be associated with the liner to assist with perspiration removal.

FIGS. 1A and 1B illustrate an exemplary embodiment of a polymeric prosthetic liner perspiration removal system 100. System 100 may include a prosthetic liner 102 having a polymeric body 104 with an open proximal 106 end and a substantially closed distal end 108, and an exterior surface that may be covered with fabric 110. A section of a perspiration wicking fabric 112 may also be present along liner 102 interior for the purposes of collecting and preferably, wicking collected perspiration toward distal end 108 of liner 102. Interior perspiration wicking fabric 112 may extend proximally for some distance from closed distal end 108 of liner 102. Perspiration wicking fabric 112 may be provided, in this particular example, in the form of a distal fabric cup 114 that mimics the general shape of liner 102 in the area where perspiration wicking fabric 112 is located. Liner 102 may be configured for placement over a residual limb 116.

Perspiration wicking fabric cup 114 may be partially covered on its proximal side by an elastomeric barrier layer 118, such that a portion of perspiration wicking fabric 112 may remain exposed for collecting and wicking away perspiration produced by residual limb 116 to which it will be donned. Elastomeric barrier layer 118 may be provided as a barrier between residual limb 116 and perspiration wicking fabric 112 when liner 102 is donned and used by an amputee. Elastomeric barrier layer 118 may provide more comfort than would otherwise be afforded by placing residual limb 116 in direct contact with perspiration wicking fabric 112 and, in combination with perspiration wicking fabric 112, may also prevent the tissue of residual limb 116 from being drawn into a port 120 as previously described. In one embodiment, system 100 may lack elastomeric barrier layer 118. Both perspiration wicking fabric 112 and elastomeric barrier layer 118 may vary in size, and the size of elastomeric barrier layer 118 with respect to the size of perspiration wicking fabric 112 may also vary so as to cover more or less of perspiration wicking fabric 112.

Elastomeric barrier layer 118 may be attached to perspiration wicking fabric 112 or may be a separate element, such as an independent gel cup. Any of the embodiments of system 100, including those exemplary embodiments described below, may be formed as molded composite structures where elastomeric barrier layer 118, perspiration wicking fabric 112, liner polymeric body 104 and exterior liner fabric 110 are all molded together to form a prosthetic liner of unitary construction. Likewise, any embodiment of system 100 may employ perspiration wicking fabric 112 that contains an antimicrobial agent for preventing or inhibiting bacterial growth between the layers of perspiration wicking fabric 112 and liner 102 and/or elastomeric barrier layer 118. The use of prosthetic liner 102 lacking exterior fabric 110 is also possible in other embodiments of system 100.

Liner 102 may include port 120 distal end 108 of liner 102. Port 120 may pass through liner polymeric body 104 and liner fabric 110. Perspiration collected by perspiration wicking fabric 112 may be expelled by compression of elastomeric barrier layer 118, including for example a gel cup, and underlying perspiration wicking fabric 112 during amputee ambulation. Alternatively, or additionally, perspiration may be withdrawn under vacuum from perspiration wicking fabric 112 through port 120. Thus, port 120 may be a perspiration expulsion port, or a vent.

FIGS. 2A and 2B illustrate an exemplary embodiment of a polymeric prosthetic liner perspiration removal system 200. System 200 may include a prosthetic liner 202 having a polymeric body 204 with an open proximal 206 end and a substantially closed distal end 208, and an exterior surface that may be covered with fabric 210. A section of a perspiration wicking fabric 212 may also be present along liner 202 interior for the purposes of collecting and preferably, wicking collected perspiration toward distal end 208 of liner 202. Interior perspiration wicking fabric 212 may extend proximally for some distance from closed distal end 208 of liner 202. Perspiration wicking fabric 212 may be provided, in this particular example, in the form of a distal fabric cup 214 that mimics the general shape of liner 202 in the area where perspiration wicking fabric 212 is located. Liner 202 may be configured for placement over a residual limb 216.

Perspiration wicking fabric cup 214 may be partially covered on its proximal side by an elastomeric barrier layer 218, such that a portion of perspiration wicking fabric 212 may remain exposed for collecting and wicking away perspiration produced by residual limb 216 to which it will be donned. Elastomeric barrier layer 218 may be provided as a barrier between residual limb 216 and perspiration wicking fabric 212 when liner 202 is donned and used by an amputee. Liner 202 may include a port 220 oriented in distal end 208.

In one embodiment, perspiration wicking fabric 212 may be designed in a manner that permits it to be pulled through port 220 so as to extend some distance externally of liner 202. In another embodiment, a separate piece of fabric may be passed through port 220 the exterior of liner 202 to contact perspiration wicking fabric 212 while also protruding from port 220. Such an arrangement may guarantee that port 220 will remain open and also provide a specific perspiration expulsion path through liner 202.

FIGS. 3A and 3B illustrate an exemplary embodiment of a polymeric prosthetic liner perspiration removal system 300. System 300 may include a prosthetic liner 302 having a polymeric body 304 with an open proximal 306 end and a substantially closed distal end 308, and an exterior surface that may be covered with fabric 310. A section of a perspiration wicking fabric 312 may also be present along liner 302 interior for the purposes of collecting and preferably, wicking collected perspiration toward distal end 308 of liner 302. Interior perspiration wicking fabric 312 may extend proximally for some distance from closed distal end 308 of liner 302. Perspiration wicking fabric 312 may be provided, in this particular example, in the form of a distal fabric cup 314 that mimics the general shape of liner 302 in the area where perspiration wicking fabric 312 is located. Liner 302 may be configured for placement over a residual limb 316.

Perspiration wicking fabric cup 314 may be partially covered on its proximal side by an elastomeric barrier layer 318, such that a portion of perspiration wicking fabric 312 may remain exposed for collecting and wicking away perspiration produced by residual limb 316 to which it will be donned. Elastomeric barrier layer 318 may be provided as a barrier between residual limb 316 and perspiration wicking fabric 312 when liner 302 is donned and used by an amputee. Liner 302 may include a port 320 oriented in distal end 308.

Liner 302 may include an exterior polymeric material 322 oriented on distal end 308, on the exterior of fabric 310. Exterior polymeric material 322 may be a polymeric or similar material.

Exterior polymeric material 322 may be substantially disc or circular in shape such that, due to the contributing profile of the distal end of the liner, exterior polymeric material 322 will appear substantially umbrella-like in shape. Exterior polymeric material 322 may be formed from a number of materials, such as, without limitation, a urethane-based material. Exterior polymeric material 322, which may be in an umbrella-like shape, may be useful to generally reinforce distal end 308 of the liner 302, and/or may act to reinforce the exterior liner fabric 310 in the area of port 320. Port 320 may extend through exterior polymeric material 322 in addition to passing through liner polymeric body 304 and fabric 310.

FIGS. 4A and 4B illustrate an exemplary embodiment of a polymeric prosthetic liner perspiration removal system 400. System 400 may include a prosthetic liner 402 having a polymeric body 404 with an open proximal 406 end and a substantially closed distal end 408, and an exterior surface that may be covered with fabric 410. A section of a perspiration wicking fabric 412 may also be present along liner 402 interior for the purposes of collecting and preferably, wicking collected perspiration toward distal end 408 of liner 402. Interior perspiration wicking fabric 412 may extend proximally for some distance from closed distal end 408 of liner 402. Perspiration wicking fabric 412 may be provided, in this particular example, in the form of a distal fabric cup 414 that mimics the general shape of liner 402 in the area where perspiration wicking fabric 412 is located. Liner 402 may be configured for placement over a residual limb 416.

Perspiration wicking fabric cup 414 may be partially covered on its proximal side by an elastomeric barrier layer 418, such that a portion of perspiration wicking fabric 412 may remain exposed for collecting and wicking away perspiration produced by residual limb 416 to which it will be donned. Elastomeric barrier layer 418 may be provided as a barrier between residual limb 416 and perspiration wicking fabric 412 when liner 402 is donned and used by an amputee. Liner 402 may include a port 420 oriented in distal end 408.

Liner 402 may include an exterior polymeric material 422 oriented on distal end 408, on the exterior of fabric 410. Exterior polymeric material 422 may be a polymeric or similar material.

In one embodiment, exterior polymeric material 422 may include a removable cap 424 oriented in an exterior polymeric material 422 portion of port 420. Cap 424 may be substantially hollow, such that perspiration may pass therethrough. Cap 424 may function primary to ensure that port 420 in liner 402 remains open during use of liner 402. Cap 424 may include a porous plug 426 that resides in the area of polymeric body 404 when cap 424 and plug 426 are installed. Plug 426 may help to ensure that port 420 in liner 402 remains open, while also transferring perspiration from the perspiration wicking fabric 412 through port 420 and cap 424.

FIGS. 5A and 5B illustrate an exemplary embodiment of a polymeric prosthetic liner perspiration removal system 500. System 500 may include a prosthetic liner 502 having a polymeric body 504 with an open proximal 506 end and a substantially closed distal end 508, and an exterior surface that may be covered with fabric 510. A section of a perspiration wicking fabric 512 may also be present along liner 502 interior for the purposes of collecting and preferably, wicking collected perspiration toward distal end 508 of liner 502. Interior perspiration wicking fabric 512 may extend proximally for some distance from closed distal end 508 of liner 502. Perspiration wicking fabric 512 may be provided, in this particular example, in the form of a distal fabric cup 514 that mimics the general shape of liner 502 in the area where perspiration wicking fabric 512 is located. Liner 502 may be configured for placement over a residual limb 516.

Perspiration wicking fabric cup 514 may be partially covered on its proximal side by an elastomeric barrier layer 518, such that a portion of perspiration wicking fabric 512 may remain exposed for collecting and wicking away perspiration produced by residual limb 516 to which it will be donned. Elastomeric barrier layer 518 may be provided as a barrier between residual limb 516 and perspiration wicking fabric 512 when liner 502 is donned and used by an amputee. Liner 502 may include a port 520 oriented in distal end 508.

Liner 502 may include an exterior polymeric material 522 oriented on distal end 508, on the exterior of fabric 510. Exterior polymeric material 522 may be a polymeric or similar material. A porous plug 526 may be oriented n port 520 in the area of polymeric body 504.

In one embodiment, system 500 may include a connecting element 528, which may replace removable cap 424 described above in reference to FIGS. 4A and 4B. Connecting element 528 may include a locking pin receiving element. At least one of connecting element 528 or the locking pin receiving element may be in the form of a threaded T-nut having an axial bore passing therethrough. Any of a variety of connecting elements may be used as connecting element 528, including any of a variety of common fasteners.

A hollow locking pin 530 may be connected to connecting element 528. The bore o f connecting element 528, in combination with the hollow interior of locking pin 530, may provide a passageway by which perspiration may be removed from the interior of liner 502. This construction may result in a locking liner with perspiration removal capability. Porous plug 526 may still be present, and connecting element 528 and/or locking pin 530 may also include a one-way valve 532 that may only permit passage of perspiration in a direction away from liner 502 and through hollow locking pin 530. Valve 532 ensures that perspiration can be removed from liner 502 while preventing the reverse uptake of perspiration.

FIGS. 6A-6D illustrate an exemplary embodiment of a polymeric prosthetic liner perspiration removal system 600. System 600 may include a prosthetic liner 602 having a polymeric body 604 with an open proximal 606 end and a substantially closed distal end 608, and an exterior surface that may be covered with fabric 610. A section of a perspiration wicking fabric 612 may also be present along liner 602 interior for the purposes of collecting and preferably, wicking collected perspiration toward distal end 608 of liner 602. Interior perspiration wicking fabric 612 may extend proximally for some distance from closed distal end 608 of liner 602. Perspiration wicking fabric 612 may be provided, in this particular example, in the form of a distal fabric cup 614 that mimics the general shape of liner 602 in the area where perspiration wicking fabric 612 is located. Liner 602 may be configured for placement over a residual limb 616.

Perspiration wicking fabric cup 614 may be partially covered on its proximal side by an elastomeric barrier layer 618, such that a portion of perspiration wicking fabric 612 may remain exposed for collecting and wicking away perspiration produced by residual limb 616 to which it will be donned. Elastomeric barrier layer 618 may be provided as a barrier between residual limb 616 and perspiration wicking fabric 612 when liner 602 is donned and used by an amputee. Liner 602 may include a port 620 oriented in distal end 608.

Liner 602 may include an exterior polymeric material 622 oriented on distal end 608, on the exterior of fabric 610. Exterior polymeric material 622 may be a polymeric or similar material.

Liner 602 at distal end 608 may have an increased thickness in comparison to previously-described embodiments. A bladder-type pump 634 may be oriented in the thickened portion of polymeric body 604 of liner 602. Pump 634 may be molded into polymeric body 604. Pump 634 may be removable from polymeric body 604.

Pump 634 may include a proximal inlet 636 and a distal outlet 638, each of which may be in fluid communication with port 620. Perspiration from perspiration wicking fabric 612 may be drawn into pump 634 through proximal inlet 636 and expelled via distal outlet 638 through the outlet of port 620.

Operation of pump 634 may occur during amputee ambulation while wearing liner 602. More specifically, a bladder 640 may be compressed (as shown in FIGS. 6C and 6D) under the amputee's weight during the gait cycle, and then may re-inflate (as shown in FIGS. 6A and 6B) when the amputee's weight is removed as the foot is lifted. This recurrent deflation and re-inflation may cause a pumping action that helps to draw perspiration out of perspiration wicking fabric 612 and to expel the perspiration through the outlet of port 620 to which pump 634 is fluidly connected. Bladder 640 may be operatively connected to port 620, and may operate as a pump during ambulation of residual limb 616.

Several one-way (check) valves may be associated with pump 634. For example, a first check valve 642 may be associated with proximal inlet 636 and a second check valve 644 may be associated with distal outlet 638 to ensure that air and perspiration can only travel through pump 634 in a direction that draws perspiration from perspiration wicking fabric 612 and expels it from port 620 during pump 634 operation. A third check valve 646 may be associated with an intake passageway/port 648 that permits outside air to be drawn into pump 634 during operation so as to permit re-inflation after pump 634 is compressed.

FIGS. 7A-7D illustrate an exemplary embodiment of a polymeric prosthetic liner perspiration removal system 700. System 700 may include a prosthetic liner 702 having a polymeric body 704 with an open proximal 706 end and a substantially closed distal end 708, and an exterior surface that may be covered with fabric 710. A section of a perspiration wicking fabric 712 may also be present along liner 702 interior for the purposes of collecting and preferably, wicking collected perspiration toward distal end 708 of liner 702. Interior perspiration wicking fabric 712 may extend proximally for some distance from closed distal end 708 of liner 702. Perspiration wicking fabric 712 may be provided, in this particular example, in the form of a distal fabric cup 714 that mimics the general shape of liner 702 in the area where perspiration wicking fabric 712 is located. Liner 702 may be configured for placement over a residual limb 716.

Perspiration wicking fabric cup 714 may be partially covered on its proximal side by an elastomeric barrier layer 718, such that a portion of perspiration wicking fabric 712 may remain exposed for collecting and wicking away perspiration produced by residual limb 716 to which it will be donned. Elastomeric barrier layer 718 may be provided as a barrier between residual limb 716 and perspiration wicking fabric 712 when liner 702 is donned and used by an amputee. Liner 702 may include a port 720 oriented in distal end 708.

Liner 702 may include an exterior polymeric material 722 oriented on distal end 708, on the exterior of fabric 710. Exterior polymeric material 722 may be a polymeric or similar material.

Exterior polymeric material 722 may have a greater thickness than in previous embodiments. A bladder-type pump 734 may be oriented in the thickened portion of exterior polymeric material 722. Pump 734 may be molded into exterior polymeric material 722. Pump 734 may be removable from exterior polymeric material 722.

Pump 734 may include a proximal inlet 736 and a distal outlet 738, each of which may be in fluid communication with port 720. Operation of pump 734 may be substantially similar to pump 634 described above and illustrated in FIGS. 6A-6D. A bladder 740 may be compressed (as shown in FIGS. 7C and 7D) under the amputee's weight during the gait cycle, and then may re-inflate (as shown in FIGS. 7A and 7B) when the amputee's weight is removed as the foot is lifted.

Several one-way (check) valves may be associated with pump 734. For example, a first check valve 742 may be associated with proximal inlet 736 and a second check valve 744 may be associated with distal outlet 738 to ensure that air and perspiration can only travel through pump 734 in a direction that draws perspiration from perspiration wicking fabric 712 and expels it from port 720 during pump 734 operation. A third check valve 746 may be associated with an intake passageway/port 748 that permits outside air to be drawn into pump 734 during operation so as to permit re-inflation after pump 734 is compressed.

FIGS. 8A-8B illustrate an exemplary embodiment of a polymeric prosthetic liner perspiration removal system 800. System 800 may include a liner 802 including a polymeric body 804, a proximal end 806, a distal end 808, and an optional fabric 810 covering the exterior of liner 802. A section of a perspiration wicking fabric 812 may also be present along liner 802 interior for the purposes of collecting and preferably, wicking collected perspiration toward distal end 808 of liner 802. Perspiration wicking fabric 812 may be provided, in this particular example, in the form of a distal fabric cup 814 that mimics the general shape of liner 802 in the area where perspiration wicking fabric 812 is located. Liner 802 may be configured for placement over a residual limb 816.

Perspiration wicking fabric cup 814 may be partially covered on its proximal side by an elastomeric barrier layer 818, such that a portion of perspiration wicking fabric 812 may remain exposed for collecting and wicking away perspiration produced by residual limb 816 to which it will be donned. Liner 802 may include a port 820 oriented in distal end 808.

A powered vacuum pump 850 may be placed in a housing 852 that is attached to a distal end of an associated prosthetic socket 854. Vacuum pump 850 may be operatively connected to port 820. A vacuum port 856 may pass through the wall of socket 854 at the distal end of socket 854. When liner 802 is properly donned to residual limb 816 and the liner-covered residual limb 816 is placed in socket 854, port 820 in liner 802 may be in communication with vacuum port 856 in socket 854.

A perspiration evacuation port 858 may be located in vacuum pump housing 852 so as to be in communication with vacuum port 856 in socket 854. The perspiration evacuation port 858 may be connected to a vacuum passageway 860 that may lead to vacuum pump 850, such that operation of vacuum pump 850 will draw perspiration out of perspiration wicking fabric 812 of liner 802 via perspiration expulsion port 858 and vacuum port 856.

Vacuum pump 850 may be an electrically-powered vacuum pump. A reservoir 862 for collecting evacuated perspiration may be located in the housing and in communication with vacuum pump 850 so that evacuated perspiration may be deposited in reservoir 862 for subsequent removal. When present, such a reservoir may be covered with a waterproof membrane 864 that causes perspiration to be retained within reservoir 862 but allows air to pass therethrough for proper operation of vacuum pump 850. Alternatively, perspiration may be expelled from vacuum pump 850 to the atmosphere or to some other location other than reservoir 862.

FIGS. 9A-9B illustrate an exemplary embodiment of a polymeric prosthetic liner perspiration removal system 900. System 900 may include a liner 902 including a polymeric body 904, a proximal end 906, a distal end 908, and an optional fabric 910 covering the exterior of liner 902. A section of a perspiration wicking fabric 912 may also be present along liner 902 interior for the purposes of collecting and preferably, wicking collected perspiration toward distal end 908 of liner 902. Perspiration wicking fabric 912 may be provided, in this particular example, in the form of a distal fabric cup 914. Liner 902 may be configured for placement over a residual limb 916.

Perspiration wicking fabric cup 914 may be partially covered on its proximal side by an elastomeric barrier layer 918. Liner 902 may include a port 920 oriented in distal end 908. System 900 may additionally include a prosthetic socket 954. Socket 954 may include a vent 966 oriented in a wall of socket 954. Vent 966 may include a small valve, a passageway, or a combination of these two items. Vent 966 may be configured to permit a deliberate and controlled entry of outside air into socket 954. Air entering socket 954 through be wicked along the exterior surface of liner 902 via exterior liner fabric 910. A vacuum pump (not shown) similar to vacuum pump 850 discussed above with reference to FIGS. 8A and 8B, may operatively connect to, or be integral to, system 900. The vacuum pump may be a programmable vacuum pump or may be otherwise controlled so as to normally operate only when a vacuum level within socket 954 is at or rises above some predetermined level. Therefore, the vacuum pump may not operate at all for an extended period of time if socket 954 is well sealed. While desirable from the standpoint of suspension, such a situation may not be conducive to vacuum-aided perspiration removal according to system 900. Providing the vent 966 through socket 954′s wall may allow the vacuum level within socket 954 to rise in a controlled manner and may result in periodic operation of the vacuum pump (not shown) even if socket 954 is otherwise perfectly sealed. Consequently, the vacuum pump may be used to draw out perspiration from within liner 902 regardless of how well the associated socket 954 is sealed.

Valves (not shown) could also be provided to permit different vacuum levels for drawing perspiration from liner 902 and for suspending liner 902 to socket 954. That is, a higher level of vacuum may be desirable for suspending liner 902 to socket 954 than is desirable for drawing perspiration from liner 902.

FIG. 10 illustrates an exemplary embodiment of a polymeric prosthetic liner perspiration removal system 1000. System 1000 may include a liner 1002 including a polymeric body 1004, a proximal end 1006, a distal end 1008, and an optional fabric 1010 covering the exterior of liner 1002. A section of a perspiration wicking fabric 1012 may also be present along liner 1002 interior for the purposes of collecting and preferably, wicking collected perspiration toward distal end 1008 of liner 1002. Perspiration wicking fabric 1012 may be provided, in this particular example, in the form of a distal fabric cup 1014. Liner 1002 may be configured for placement over a residual limb 1016.

Perspiration wicking fabric cup 1014 may be partially covered on its proximal side by an elastomeric barrier layer 1018. Liner 1002 may include a port 1020 oriented in distal end 1008. Liner 1002 may additionally include an exterior polymeric material (not shown). Port 120 may pass additionally extend through an exterior polymeric material, if an exterior polymeric material is present.

System 1000 may include a 3-D spacer fabric 1068 as a substitute for at least a portion of liner 1002. Spacer fabric 1068 may be oriented near proximal end 1006. In one embodiment, spacer fabric 1068 may be oriented to replace any of a variety of portions of liner 1002, including near proximal end 1006, distal end 1008, or between proximal end 1006 and distal end 1008.

3-D spacer fabric 1068 may be a special fabric that is of substantial thickness. 3-D spacer fabric 1068 may be able to provide a cushioning effect. 3-D spacer fabric 1068 may have moisture and/or air wicking capabilities. 3-D spacer fabric 1068 may form an entire upper (proximal) portion of liner 1002 (as illustrated in FIG. 10), acting as a complete substitute for polymeric body 1004 or polymeric body 1004 and fabric 1010 that normally form that portion of liner 1002. 3-D spacer fabric 1068 may form a window (not shown) or windows (not shown) in liner 1002, and may again substitute for the material(s) that would normally reside at that location(s). When one or more windows of 3-D spacer fabric 1068 are used, an overlying prosthetic socket (not shown) that is used with liner 1002 may have a matching window(s) passing therethrough to facilitate the evaporation of perspiration absorbed by 3-D fabric 1068.

FIGS. 11A and 11B illustrate an exemplary embodiment of a polymeric prosthetic liner perspiration removal system 1100. System 1100 may include a liner 1102 having a polymeric body 1104, and an optional fabric 1110. Liner 1102 may include a proximal end 1106 and a distal end 1108. A section of a perspiration wicking fabric 1112 may also be present along liner 1102 interior for the purposes of collecting and preferably, wicking collected perspiration. Perspiration wicking fabric 1112 may be provided, in this particular example, in the form of a distal fabric cup 1114. Liner 1102 may be configured for placement over a residual limb (not shown).

Perspiration wicking fabric cup 1114 may be partially covered on its proximal side by an elastomeric barrier layer 1118. Liner 1102 may include a port (not shown) for expulsion of perspiration. In one embodiment, liner 1102 does not include a port for expulsion of perspiration.

Liner 1102 may include bands of wicking material 1170. Wicking bands 1170 may be operatively connected with polymeric body 1104. Liner 1102 may include wicking bands 1170 along the interior of liner 1102, and oriented in a distal-to-proximal direction (longitudinally along liner 1102).

Wicking bands 1170 may be arranged such that the distal ends or distal sections of wicking bands 1170 are in communication with perspiration wicking fabric 1112. Wicking bands 1170 may be in contact with perspiration wicking fabric 1112. Wicking bands 1170 may be in communication with, and/or contact with, perspiration wicking fabric 1112 at distal end 1108 of liner 1102. Wicking bands 1170 may be arranged to wick perspiration from an area of perspiration wicking fabric 1112 toward proximal end of liner 1102, for expulsion into the atmosphere.

An exposed area 1172 of polymeric body 1104 may be present along the exterior of liner 1102 (that is, it is absent fabric 1110). Exposed area 1172 of polymeric body 1104 may be oriented near proximal end 1106 of liner 1102. Exposed area 1172 of polymeric body 1104 may be configured to seal with a sealing sleeve (not shown) to seal an open end of a prosthetic socket (not shown) into which the donned liner 1102 has been inserted.

In one embodiment, proximal end 1106 of liner 1102 is oriented such that wicking bands 1170 or against a residual limb (not shown). In another embodiment, proximal end 1106 of liner 1102 may be arranged such that wicking bands 1170 extend to the exterior or liner 1102. Liner 1102 may alternatively be reflected (folded over) at some point above a socket brim (not shown) of a socket (not shown), such that wicking bands 1170 are exposed to an exterior of liner 1102 and a socket (not shown).

In one embodiment, system 1100 includes a hollow pin (not shown) and a perspiration expulsion port (not shown) oriented at distal end 1108. Wicking bands 1170 may be exposed to an atmospheric pressure at proximal end 1106 of liner 1102, and consequently wicking bands 1170 may provide a wicking path to the atmosphere.

FIGS. 12A and 12B illustrate an exemplary embodiment of a polymeric prosthetic liner perspiration removal system 1200. System 1200 may include a liner 1202 having a polymeric body 1204, and an optional fabric 1210. Liner 1202 may include a proximal end 1206 and a distal end 1208. A section of a perspiration wicking fabric 1212 may also be present along liner 1202 interior for the purposes of collecting and preferably, wicking collected perspiration. Perspiration wicking fabric 1212 may be provided, in this particular example, in the form of a distal fabric cup 1214. Liner 1202 may be configured for placement over a residual limb 1216.

Perspiration wicking fabric cup 1214 may be partially covered on its proximal side by an elastomeric barrier layer 1218. Liner 1202 may include a port (not shown) for expulsion of perspiration. In one embodiment, liner 1202 does not include a port for expulsion of perspiration.

Liner 1202 may include bands of wicking material 1270. Wicking bands 1270 may be operatively connected with polymeric body 1204. Liner 1202 may include wicking bands 1270 along the interior of liner 1202, and oriented in a distal-to-proximal direction (longitudinally along liner 1202).

Wicking bands 1270 may be arranged such that the distal ends or distal sections of wicking bands 1270 are in communication with perspiration wicking fabric 1212. Wicking bands 1270 may be in contact with perspiration wicking fabric 1212. Wicking bands 1270 may be in communication with, and/or contact with, perspiration wicking fabric 1212 at distal end 1208 of liner 1202. Wicking bands 1270 may be arranged to wick perspiration from an area of perspiration wicking fabric 1212 toward proximal end of liner 1202, for expulsion into the atmosphere.

System 1200 may include a prosthetic socket 1254. A liner 1202 may be donned over residual limb 1216 and inserted into socket 1254.

Liner 1202 may include an area an exposed area 1272 of polymeric body 1204. Exposed area 1272 may be oriented near proximal end 1206 of liner 1202. Exposed area 1272 may provide a surface against which a sealing sleeve 1274 may seal when sealing sleeve 1274 is used with liner 1202 to seal the open end of prosthetic socket 1254 into which the donned liner 1202 has been inserted. This configuration may be employed for example, when perspiration is not an issue.

If/when perspiration does become an issue, proximal end 1206 of liner 1202 may be reflected (folded over) at a reflection point 1276 above socket 1254′s brim, as is illustrated in FIG. 12B. This reflection of proximal end 1206 of liner 1202 may expose wicking bands 1270. One end of sealing sleeve 1274 may then be sealed against the exterior of socket 1254, while the other end of sealing sleeve 1274 seals against the skin of the amputee's residual limb 1216. Sealing sleeve 1274 does not seal against the exposed wicking bands 1270.

While not shown in FIGS. 12A and 12B, one embodiment of system 1200 could also include a vacuum pump (not shown), such as an electrically powered vacuum pump. For example, vacuum pump 850 and housing 852 of FIGS. 8A and 8B may be used with an appropriately modified socket. In any event, the socket used with such a liner may include a vacuum port that passes therethrough.

When a vacuum pump is operated with liner 1202 in the non-reflected orientation of FIG. 12A and sealing sleeve 1274 sealed against the exterior of prosthetic socket 1254, and exposed area 1272 at proximal end 1206 of liner 1202, the vacuum produced by the vacuum pump (not shown) may serve to evacuate socket 1254 through the vacuum port (not shown), thereby drawing liner 1202 against the interior socket wall and producing a suction suspension of an associated prosthesis. However, when such a vacuum pump is operated with liner 1202 in the reflected orientation of FIG. 12B and sealing sleeve 1274 sealed against the exterior of prosthetic socket 1254 and the skin of the amputee's residual limb 1216 as shown, a vacuum pathway may be created between the vacuum port (not shown) in socket 1254 and the reflected proximal end 1206 of liner 1202. Consequently, the vacuum produced by the vacuum pump (not shown) may act on the exposed area of wicking bands 1270, which serves to draw perspiration from perspiration wicking fabric 1212 upward (proximally) through wicking bands 1270 where it may be removed from the open end of liner 1202.

In one embodiment, wicking bands like those described above may be used with a liner that includes a perspiration expulsion port in the distal end thereof. Such an embodiment may not be used in a vacuum suspension application because the vacuum pump may run continuously. However, such a construction may be used in a suction suspension construction or possibly a locking suspension construction that includes a hollow pin. In such an embodiment, the wicking bands may help to move perspiration more readily since the wicking bands are generally exposed to atmospheric pressure at the proximal edge of the liner. Consequently, the wicking bands may provide a better wicking path than the fabric cup when used in such embodiments.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “substantially” is used in the specification or the claims, it is intended to take into consideration the degree of precision available or prudent in manufacturing. To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. As used in the specification and the claims, the singular forms “a,” “an,” and “the” include the plural. Finally, where the term “about” is used in conjunction with a number, it is intended to include±10% of the number. In other words, “about 10” may mean from 9 to 11.

As stated above, while the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

1. A system for removing perspiration from an interior of a donned polymeric prosthetic liner, comprising:
a prosthetic liner having a polymeric body, the polymeric body having an open proximal end for allowing the introduction of a residual limb, a closed or essentially closed distal end opposite the proximal end, an interior, and an exterior;
a perspiration wicking fabric disposed along the interior of the polymeric body at the distal end thereof;
an elastomeric barrier layer disposed along a portion of a proximal side of the perspiration wicking fabric; and
a port located at the distal end of the polymeric body and passing through the polymeric body to expose an area of the perspiration wicking fabric.

2. The system of claim 1, further comprising a fabric material optionally attached to and covering all or a majority of the exterior of the polymeric body.

3. The system of claim 2, further comprising an exterior polymeric material oriented on the fabric material at the distal end of the prosthetic liner, wherein the port extends through the exterior polymeric material.

4. The system of claim 3, further comprising a removable cap oriented in an exterior polymeric material portion of the port.

5. The system of claim 1, further comprising a porous plug oriented in a polymeric body portion of the port.

6. The system of claim 1, further comprising a vacuum pump operatively connected to the port.

7. The system of claim 1, further comprising a compressible bladder operatively connected to the port, wherein the bladder operates as a pump during ambulation of the residual limb.

8. The system of claim 1, further comprising a 3-D fabric material replacing at least a portion of the polymeric body of the prosthetic liner.

9. The system of claim 1, wherein at least a portion of the perspiration wicking fabric extends through the port.

10. The system of claim 1, further comprising a connecting element oriented on the distal end of the prosthetic liner.

11. The system of claim 10, further comprising a hollow locking pin connected to the connecting element, and including a passageway by which a perspiration may travel from the interior of the polymeric body.

12. The system of claim 1, further comprising a prosthetic socket, wherein a vent is oriented in a wall of the prosthetic socket.

13. The system of claim 1, further comprising at least one wicking band having a distal end in communication with the perspiration wicking fabric.

14. A system for removing perspiration from an interior of a donned polymeric prosthetic liner, comprising:
a prosthetic liner having a polymeric body, the polymeric body having an open proximal end for allowing the introduction of a residual limb, a closed or essentially closed distal end opposite the proximal end, an interior, and an exterior;
a perspiration wicking fabric disposed along the interior of the polymeric body at the distal end thereof;
an elastomeric barrier layer disposed along a portion of a proximal side of the perspiration wicking fabric; and
at least one wicking band having a distal end, the distal end being in communication with the perspiration wicking fabric.

15. The system of claim 14, further comprising a fabric material attached to and covering all or a majority of an exterior of the polymeric body;

16. The system of claim 14, wherein the at least one wicking band is oriented longitudinally within the prosthetic liner.

17. The system of claim 14, wherein the at least one wicking band is exposed to an atmospheric pressure at the proximal end of the prosthetic liner.

18. The system of claim 14, further comprising a port located at the distal end of the polymeric body and passing through the polymeric body and the fabric material to expose an area of the perspiration wicking fabric.

19. The system of claim 14, wherein the at least one wicking band is exposed to the exterior of the prosthetic liner.

20. The system of claim 14, wherein the proximal end of the prosthetic liner is reflected.
